(19) United States
(12) Patent Application Publication
(10) Pub. No.: US 2018/0235684 A1
(43) Pub. Date: Aug. 23, 2018

(54) Self-Retaining Screwdriver

(71) Applicant: Nexus Spine, LLC, Salt Lake City, UT (US)

(72) Inventors: David Hawkes, Pleasant Grove, UT (US); Peter Halverson, Draper, UT (US)

(21) Appl. No.: 15/795,240

(22) Filed: Oct. 26, 2017

Related U.S. Application Data
(60) Provisional application No. 62/413,406, filed on Oct. 26, 2016.

(51) Int. Cl. A61B 17/88; A61B 17/70

(52) U.S. Cl. CPC A61B 17/8888; A61B 17/7082

(57) ABSTRACT

A self-retaining screwdriver includes an elongate inner driver rod and an outer sleeve disposed about the inner driver rod and having a distal end, the distal end having a plurality of fingers. Each of the fingers includes a retention feature. The retention feature may be formed as an area of thickening of the finger, whereby an effective diameter of the outer sleeve at the retention features of the fingers is increased. When the inner driver rod is disposed within the outer sleeve with a distal portion of the inner driver rod extended as far as the retention feature, the presence of the inner driver rod prevents the fingers from being radially displaced inwardly. When the inner driver rod is at least partially withdrawn, the distal portion of the inner driver rod does not extend to a distal end of the fingers, and the fingers can be radially displaced inwardly.

### CROSS-REFERENCE TO RELATED APPLICATIONS

This application claims the benefit of U.S. Provisional Application No. 62/413,406, filed Oct. 26, 2016.

### BACKGROUND OF THE INVENTION

### 1. Field of the Invention

The present invention relates to screwdrivers for surgical procedures, and more particularly to self-retaining screwdrivers

### 2. Background and Related Art

In many instances, it may be desirable to securely hold a fastener to a tool while the fastener is placed and fastened. For example, in a surgical environment it may be desirable to hold a fastener such as a surgical screw to a fastening tool such as a screwdriver while the fastener is secured (e.g., screwed) at the surgical site, such as into bone. Depending on the surgical site and the type of procedure, such as a minimally invasive procedure, temporary securing of the fastener to the tool may ensure that the fastener is not lost within the surgical site.

Several existing methods have been used to secure fasteners such as screws to tools such as screwdrivers. In one example, the tool or fastener may be magnetized. This method is not generally used in surgical environments as both the tool and the fastener must be ferrous, and while some surgical screws are formed of stainless steel, the magnetic response of stainless steel is generally insufficient for reliable use to secure fastener to tool. Additionally, magnetic tools are prone to accumulation of ferrous debris that may be incompatible with sterilization procedures and/or the surgical site. Accordingly, magnetic tools are not typically used as systems for securing tool to fastener in surgical applications.

Another mechanism that can be used to secure tool to fastener (screwdriver to screw) is an external securing feature that surrounds all or a portion of the tool end of the fastener. For example, elastic sleeves may be affixed to the end of the tool, and the fastener is inserted into the elastic sleeve until respective driving features engage. Other external securing features can include movable clips that grab or otherwise releasably secure the fastener (e.g., by a screw head) to the tool. External features generally have an effect of obscuring the view of the fastener, which in the surgical field can lead to non-optimal placement of the surgical fastener (e.g., surgical screw) in the bone. Accordingly, while external securing features are sometimes used in the surgical environment, their use is not ideal.

An alternate mechanism for securing tool to fastener is an internal polymeric feature that is compressed between the tool and the fastener when a driving portion of the tool is inserted into a driving feature of the fastener. Most typically, the polymeric feature is located on a driving surface of the tool. While polymeric features are internal to the tool-fastener interface and avoid the problem of obscuring the surgeon's view of the fastener, the polymeric features tend to wear quickly and often do not pass through sterilization procedures effectively. Such features may also lack sufficient retention force for some applications such that the fastener may not be effectively retained on the tool in all situations, especially as the polymeric feature wears.

Accordingly, existing fastener retention mechanisms fail to adequately address all fastener retention needs. The need for improved fastener retention is especially felt in the surgical field, particularly with respect to minimally invasive surgery.

### BRIEF SUMMARY OF THE INVENTION

Implementation of the invention provides self-retaining screwdrivers and methods for using the self-retaining screwdrivers. An exemplary self-retaining screwdriver includes an elongate inner driver rod and an outer sleeve adapted to be disposed about the inner driver rod and including a distal end, the distal end of the outer sleeve including a plurality of fingers separated by longitudinal slots in the outer sleeve.

Each of the fingers may include a retention feature. The retention feature may be formed as an area of thickening of the finger, whereby an effective diameter of the outer sleeve at the retention features of the fingers is increased. When the inner driver rod is disposed within the outer sleeve with a distal portion of the inner driver rod extended as far as the retention feature, the presence of the inner driver rod prevents the fingers from being radially displaced inwardly. When the inner driver rod is at least partially withdrawn proximally within the outer sleeve such that the distal portion of the inner driver rod does not extend to a distal end of the fingers, the fingers can be radially displaced inwardly, effectively decreasing the diameter of the outer sleeve at the retention features of the fingers.

The inner driver rod may include a distal end having a driver feature adapted to engage a screw and to transfer rotational torque from the inner driver rod to the screw. The outer sleeve may include a longitudinal alignment slot, and the inner driver rod may include a protrusion adapted to be received within the longitudinal alignment slot of the outer sleeve. The protrusion and the alignment slot may permit transmission of rotational torque from the inner driver rod to the outer sleeve or from the outer sleeve to the inner driver rod. The self-retaining screwdriver may include a detent that limits unintentional retraction of the inner driver rod from the outer sleeve. The elongate inner driver rod may be hollow to permit passage of a guide wire therethrough.

Another exemplary self-retaining screwdriver includes an elongate inner driver rod and an outer sleeve adapted to be disposed about the inner driver rod and having a distal end, the distal end of the outer sleeve having a plurality of fingers separated by longitudinal slots in the outer sleeve, and wherein each of the fingers includes a retention feature.

The retention feature may be formed as an area of thickening of the finger, whereby an effective diameter of the outer sleeve at the retention features of the fingers is increased. When the inner driver rod is disposed within the outer sleeve with a distal portion of the inner driver rod extended as far as the retention feature, the presence of the inner driver rod prevents the fingers from being radially displaced inwardly. When the inner driver rod is at least partially withdrawn proximally within the outer sleeve such that the distal portion of the inner driver rod does not extend to a distal end of the fingers, the fingers can be radially displaced inwardly, effectively decreasing the diameter of the outer sleeve at the retention features of the fingers.

The inner driver rod may include a distal end having a driver feature adapted to engage a screw and to transfer rotational torque from the inner driver rod to the screw. The outer sleeve may include a longitudinal alignment slot, and the inner driver rod may include a protrusion adapted to be received within the longitudinal alignment slot of the outer sleeve. The protrusion and the alignment slot may permit transmission of rotational torque from the inner driver rod to the outer sleeve or from the outer sleeve to the inner driver rod. The self-retaining screwdriver may include a detent that limits unintentional retraction of the inner driver rod from the outer sleeve. The elongate inner driver rod may be hollow to permit passage of a guide wire therethrough.

A method for preparing a self-retaining screw driver with a surgical screw retained thereon ready for use in a surgical procedure includes steps of providing an outer sleeve having a distal end having a plurality of fingers separated by longitudinal slots in the outer sleeve, advancing the outer sleeve into a partially spherical receptacle of a surgical screw, whereby the fingers are temporarily and elastically forced radially inward as the surgical screw is received on the outer sleeve, and advancing an inner sleeve into the outer sleeve until a distal end of the inner sleeve is disposed within the fingers, thereby forcing the fingers radially outwardly and preventing the fingers from being displaced radially inwardly, thereby retaining the surgical screw on the outer sleeve. The inner sleeve may be advanced further within the outer sleeve until a tip of the distal end of the inner sleeve protrudes beyond the outer sleeve and mates with and engages a driving feature of the surgical screw located more distal than the partially spherical receptacle.

### BRIEF DESCRIPTION OF THE SEVERAL VIEWS OF THE DRAWINGS

The objects and features of the present invention will become more fully apparent from the following description and appended claims, taken in conjunction with the accompanying drawings. Understanding that these drawings depict only typical embodiments of the invention and are, therefore, not to be considered limiting of its scope, the invention will be described and explained with additional specificity and detail through the use of the accompanying drawings in which:

FIG. 1 shows a side view of an exemplary self-retaining screwdriver;

FIG. 2 shows an alternate side view of an exemplary self-retaining screwdriver;

FIG. 3 shows a cross-sectional view of a tip of an exemplary self-retaining screwdriver inserted into a surgical screw with an inner sleeve retracted;

FIG. 4 shows a cross-sectional view of a tip of an exemplary self-retaining screwdriver inserted into a surgical screw with an inner sleeve partially advanced;

FIG. 5 shows a cross-sectional view of a tip of an exemplary self-retaining screwdriver inserted into a surgical screw with an inner sleeve fully advanced;

FIG. 6 shows a side view of a detent engagement between an outer shaft and an inner shaft;

FIG. 7 shows a side view of a fully engaged detent as depicted in FIG. 6; and

FIG. 8 shows a cross-sectional view of a tip of an exemplary self-retaining screwdriver fully advanced in the absence of a surgical screw for use as a traditional, non-retaining, screwdriver.

### DETAILED DESCRIPTION OF THE INVENTION

A description of embodiments of the present invention will now be given with reference to the Figures. It is expected that the present invention may take many other forms and shapes, hence the following disclosure is intended to be illustrative and not limiting, and the scope of the invention should be determined by reference to the appended claims.

Embodiments of the invention provide self-retaining screwdrivers and methods for using the self-retaining screwdrivers. An exemplary self-retaining screwdriver includes an elongate inner driver rod and an outer sleeve adapted to be disposed about the inner driver rod and including a distal end, the distal end of the outer sleeve having a plurality of fingers separated by longitudinal slots in the outer sleeve.

Each of the fingers may include a retention feature. The retention feature may be formed as an area of thickening of the finger, whereby an effective diameter of the outer sleeve at the retention features of the fingers is increased. When the inner driver rod is disposed within the outer sleeve with a distal portion of the inner driver rod extended as far as the retention feature, the presence of the inner driver rod prevents the fingers from being radially displaced inwardly. When the inner driver rod is at least partially withdrawn proximally within the outer sleeve such that the distal portion of the inner driver rod does not extend to a distal end of the fingers, the fingers can be radially displaced inwardly, effectively decreasing the diameter of the outer sleeve at the retention features of the fingers.

The inner driver rod may include a distal end having a driver feature adapted to engage a screw and to transfer rotational torque from the inner driver rod to the screw. The outer sleeve may include a longitudinal alignment slot, and the inner driver rod may include a protrusion adapted to be received within the longitudinal alignment slot of the outer sleeve. The protrusion and the alignment slot may permit transmission of rotational torque from the inner driver rod to the outer sleeve or from the outer sleeve to the inner driver rod. The self-retaining screwdriver may include a detent that limits unintentional retraction of the inner driver rod from the outer sleeve. The elongate inner driver rod may be hollow to permit passage of a guide wire therethrough.

Another exemplary self-retaining screwdriver includes an elongate inner driver rod and an outer sleeve adapted to be disposed about the inner driver rod and having a distal end, the distal end of the outer sleeve having a plurality of fingers separated by longitudinal slots in the outer sleeve, and wherein each of the fingers includes a retention feature.

The retention feature may be formed as an area of thickening of the finger, whereby an effective diameter of the outer sleeve at the retention features of the fingers is increased. When the inner driver rod is disposed within the outer sleeve with a distal portion of the inner driver rod extended as far as the retention feature, the presence of the inner driver rod prevents the fingers from being radially displaced inwardly. When the inner driver rod is at least partially withdrawn proximally within the outer sleeve such that the distal portion of the inner driver rod does not extend to a distal end of the fingers, the fingers can be radially displaced inwardly, effectively decreasing the diameter of the outer sleeve at the retention features of the fingers.

The inner driver rod may include a distal end having a driver feature adapted to engage a screw and to transfer rotational torque from the inner driver rod to the screw. The outer sleeve may include a longitudinal alignment slot, and the inner driver rod may include a protrusion adapted to be received within the longitudinal alignment slot of the outer sleeve. The protrusion and the alignment slot may permit transmission of rotational torque from the inner driver rod to the outer sleeve or from the outer sleeve to the inner driver rod. The self-retaining screwdriver may include a detent that limits unintentional retraction of the inner driver rod from the outer sleeve. The elongate inner driver rod may be hollow to permit passage of a guide wire therethrough.

A method for preparing a self-retaining screw driver with a surgical screw retained thereon ready for use in a surgical procedure includes steps of providing an outer sleeve having a distal end including a plurality of fingers separated by longitudinal slots in the outer sleeve, advancing the outer sleeve into a partially spherical receptacle of a surgical screw, whereby the fingers are temporarily and elastically forced radially inward as the surgical screw is received on the outer sleeve, and advancing an inner sleeve into the outer sleeve until a distal end of the inner sleeve is disposed within the fingers, thereby forcing the fingers radially outwardly and preventing the fingers from being displaced radially inwardly, thereby retaining the surgical screw on the outer sleeve. The inner sleeve may be advanced further within the outer sleeve until a tip of the distal end of the inner sleeve protrudes beyond the outer sleeve and mates with and engages a driving feature of the surgical screw located more distal than the partially spherical receptacle.

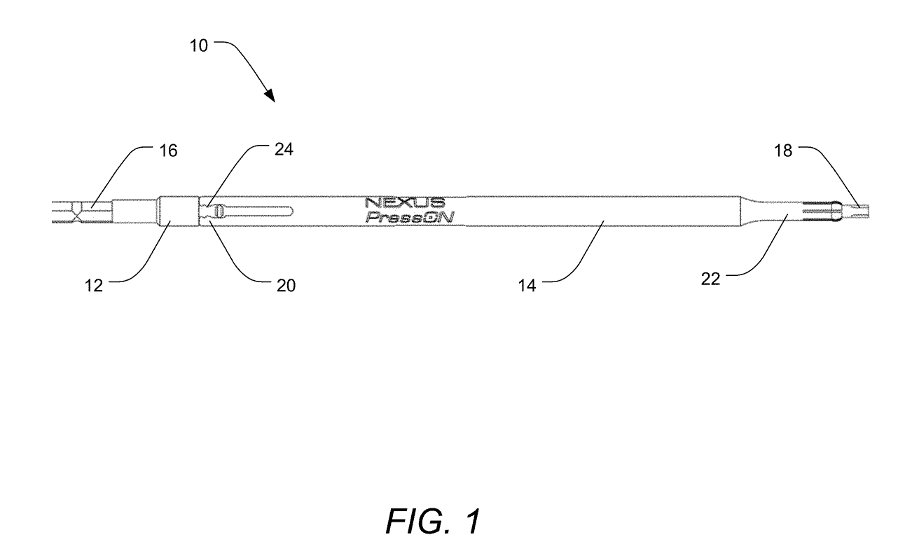

FIG. 1 shows an exemplary embodiment of a self-retaining screwdriver 10. The screwdriver 10 includes an inner sleeve 12 and an outer sleeve 14. Both of the inner sleeve 12 and the outer sleeve 14 in this example are hollow, allowing the inner sleeve 12 and the outer sleeve 14 to be disposed on a guide wire as in minimally invasive surgical procedures. In other embodiments, the inner sleeve 12 may be replaced with a solid inner shaft when it is not necessary for the screwdriver 10 to be disposed on a guide wire.

The inner sleeve 12 includes a proximal end 16 and a distal end 18. The proximal end 16 in this embodiment is adapted to be mated to a manual or powered handle, and includes features adapted to permit transfer of rotational torque from the handle to the inner sleeve 12, such as a hexagonal shank shape, a square shank shape, a star shank shape, one or more slots in the distal end 18, or the like. The distal end 18 in this embodiment is adapted to mate with a driving feature of a surgical screw such as a pedicle screw. Accordingly, a tip of the distal end 18 may have any desirable shape to mate with the driving feature of the surgical screw, such as a hexagonal shape, a square shape, a cross shape, a star shape, or any desired shape. The shape of the distal end 18 mates with a similar shape of the driving feature of the surgical screw, permitting transfer of rotational torque from the inner sleeve 12 to the surgical screw.

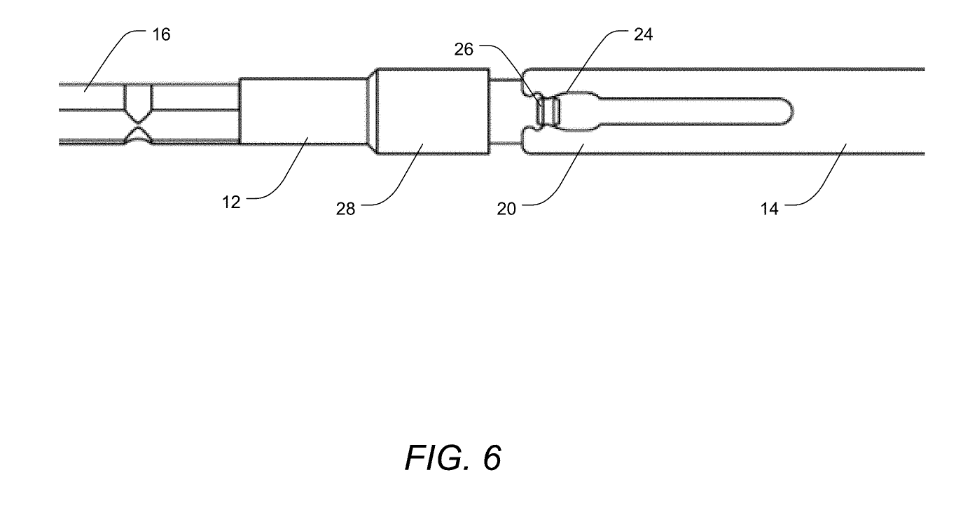

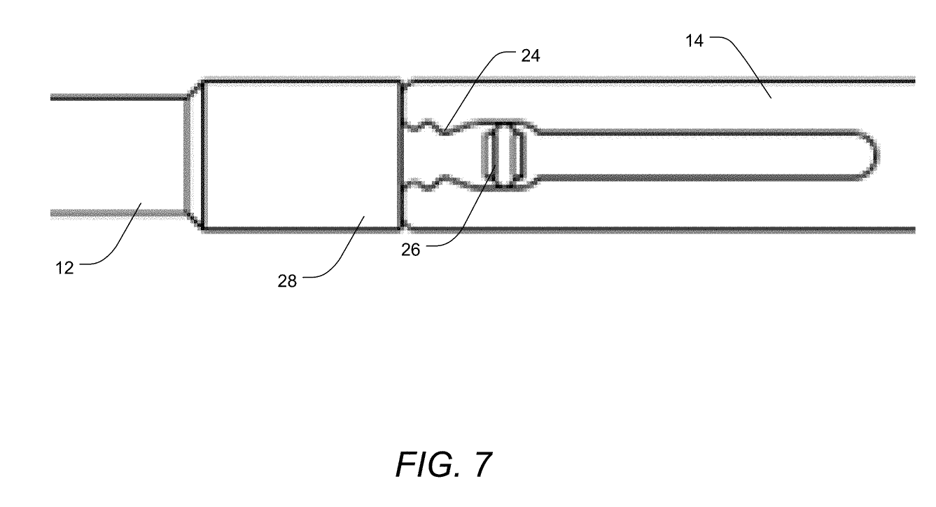

The outer sleeve 14 includes features adapted to engage with an secure the surgical screw to the screwdriver 10. The outer sleeve 14 includes a proximal end 20 and a distal end 22. The proximal end 20 receives the inner sleeve 12 therein, and the inner sleeve 12 may be advanced until the distal end 18 of the inner sleeve 12 protrudes beyond the distal end 22 of the outer sleeve 14, as shown in FIG. 1. The proximal end 20 of the outer sleeve 14 may be provided with a feature adapted to engage with a feature of the inner sleeve 12 to prevent unwanted inadvertent withdrawal of the inner sleeve 12 from the outer sleeve 14. This is illustrated as a detent 24 in FIG. 1. The detent 24 is shown in more detail in FIGS. 6-7. As the inner sleeve 12 is advanced into the outer sleeve 14, a protrusion 26 on the inner sleeve 12 catches on the detent 24 and an increased insertion force is required to push the protrusion 26 past the detent 24. Similarly, once the protrusion 26 is distal the detent 24, an increased force is required to withdraw the inner sleeve 12 and its protrusion 26 from the outer sleeve 14 and its detent 24, thereby protecting against unwanted withdrawal of the inner sleeve 12. A stop 28 may prevent advancement of the inner sleeve 12 too far into the outer sleeve 14. Detents 24 and protrusions 26 may be located at more than one circumferential on the inner sleeve 12 and the outer sleeve 14.

The detent 24 and protrusion 26 are merely one example of a feature to minimize the possibility of unwanted retraction of the inner sleeve 12 from the outer sleeve 14. In another example, friction or interference between the inner sleeve 12 and the outer sleeve 14 may serve to minimize unwanted withdrawal of the inner sleeve 12 after insertion into the outer sleeve 14.

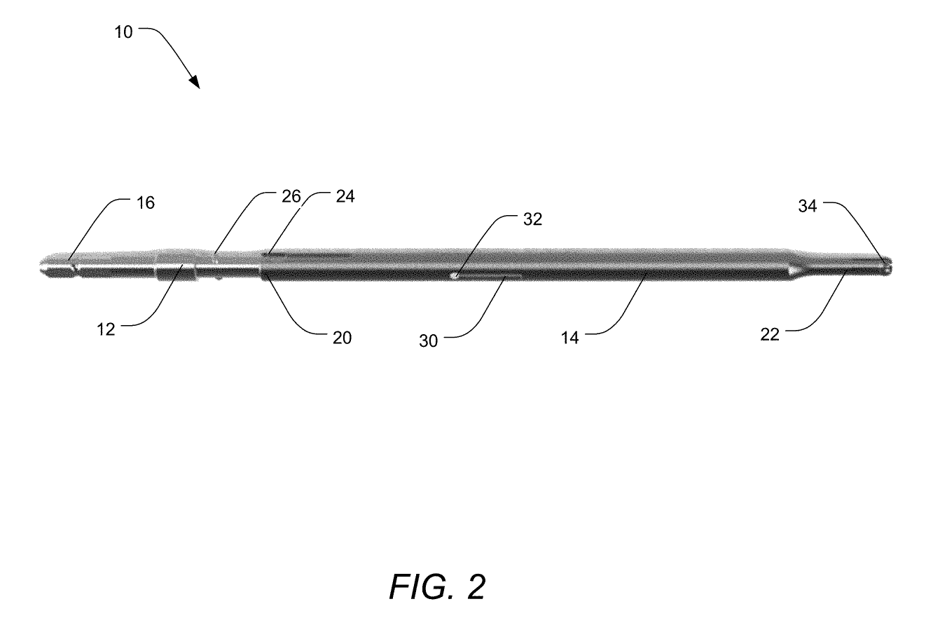

FIG. 2 shows a slightly rotated side view of the screwdriver 10, illustrating a slot 30 in the outer sleeve 14 and corresponding a pin 32 on the inner sleeve 12. The slot 30 and the pin 32 may serve any of several functions. The slot 30 and the pin 32 may serve to permit transfer of rotational torque from the inner sleeve 12 to the outer sleeve 14 in certain embodiments, as will be discussed further below. Additionally, the slot 30 and the pin 32 may keep the inner sleeve 12 and the outer sleeve 14 aligned to ensure that the protrusion 26 and the detent 24 are aligned during insertion of the inner sleeve 12 into the outer sleeve 14. In some embodiments, the pin 32 and the slot 34 may be omitted, or multiple pins 32 and slots 30 may be present as desired.

The distal end 22 of the outer sleeve 14 may be divided into a plurality of elastically deformable fingers 34 separated by longitudinal slots. At the most distal extent of the distal end 22, each finger 34 may be enlarged somewhat or protrude outward such that the most distal extent of the distal end 22 assumes a partially spherical shape. This shape may be sized and shaped to mate with a corresponding partially spherical receptacle in the surgical screw. The enlarged portion of each finger 34 serves as a retention feature. The flexibility of the fingers 34 permits the fingers 34 to flex, thereby effectively reducing the diameter of the partially spherical shape of most distal extent of the distal end 18 such that the ends of the fingers 34 can pass into the partially spherical receptacle of the surgical screw.

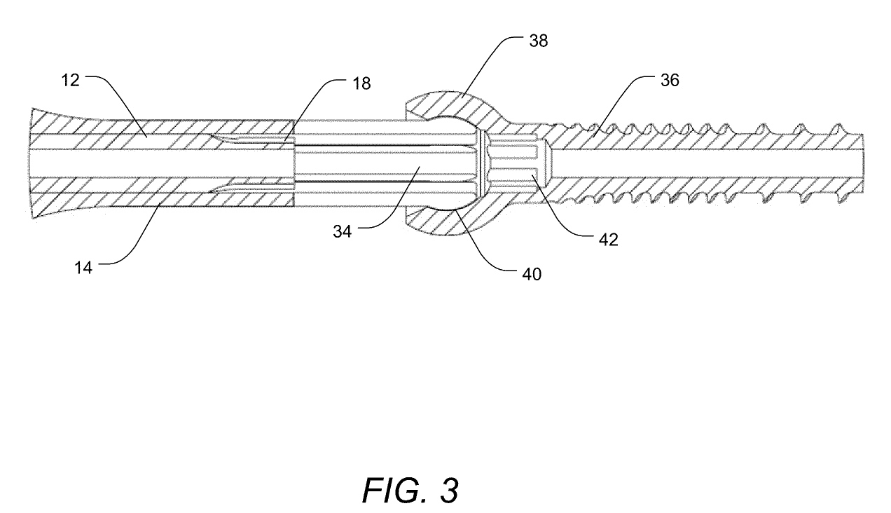

This passage of the ends of the fingers 34 into the receptacle of the surgical screw occurs when the inner sleeve 12 is at least partially withdrawn from the outer sleeve 14. This is illustrated in FIG. 3, which illustrates an exemplary surgical screw 36 having a head 38 that is generally spherical in shape and defines a generally spherical or partially spherical receptacle 40 adapted to receive the partially spherical shape formed by the plurality of fingers 34. A driving feature 42 extends distally from the receptacle 40 along the axis of the surgical screw 36, and the driving feature 42 is adapted to receive and mate with the tip of the distal end 18 of the inner sleeve 12 to permit transfer of rotational torque from the inner sleeve 12 to the surgical screw 36. The driving feature 42 and the tip of the distal end 18 of the inner sleeve 12 may accordingly be similarly sized and shaped using any desired driving system (e.g., hexagonal, star, square, triangle, tri-lobe, etc.) to provide a secure fit between the inner sleeve 12 and the surgical screw 36. If desired, the surgical screw 36 may be cannulated to permit the surgical screw to be disposed on a guide wire.

As shown in FIG. 3, however, upon insertion of the screwdriver 10 into the surgical screw 36, the inner sleeve 12 is not initially inserted into the surgical screw 10, but is instead at least partially withdrawn into the outer sleeve 14, such that the tip at the distal end 18 of the inner sleeve 12 does not engage the driving feature 42 of the surgical screw 36. In this state, the fingers 34 are permitted to flex inward to permit insertion of the screwdriver 10 into the surgical screw 36.

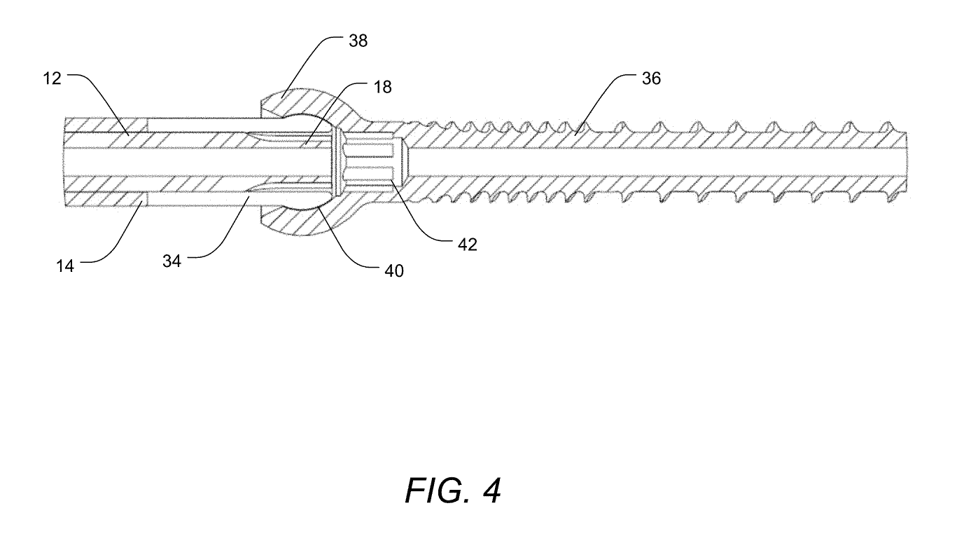

Once the outer sleeve 14 is mated with the partially spherical receptacle 40 of the surgical screw 36 such that the enlarged portion of each of the fingers 34 is within the partially spherical receptacle 40, the inner sleeve 12 may be advanced within the outer sleeve 14 until the distal end 18 of the inner sleeve is positioned within the partially spherical receptacle 40 of the surgical screw 36, as illustrated in FIG. 4. At this position, the distal end 18 of the inner sleeve 12 prevents the fingers 34 from deforming inward, and the surgical screw 36 is accordingly retained on the screwdriver 10.

In some embodiments, the outward force supplied to the fingers 34 by the advancing inner sleeve 12 may be sufficiently strong to permit driving the surgical screw 36 solely by the press or interference fit between the head 38 of the surgical screw and the fingers 34 of the outer sleeve 16. Alternatively, the partially spherical receptacle 40 may be formed with one or more ridges adapted to engage with the grooves between the fingers 34, such as to facilitate transfer of rotational torque between the outer sleeve 16 and the surgical screw 36. As still another alternative, the partially spherical receptacle 40 of the surgical screw 36 may instead be formed in a non-spherical shape having one or more surfaces capable of receiving thrust, and the fingers 34 may be correspondingly shaped to create a driving tip of the outer sleeve 14 adapted to transfer rotational torque from the driving tip to the surfaces capable of receiving thrust of the receptacle of the surgical screw 36.

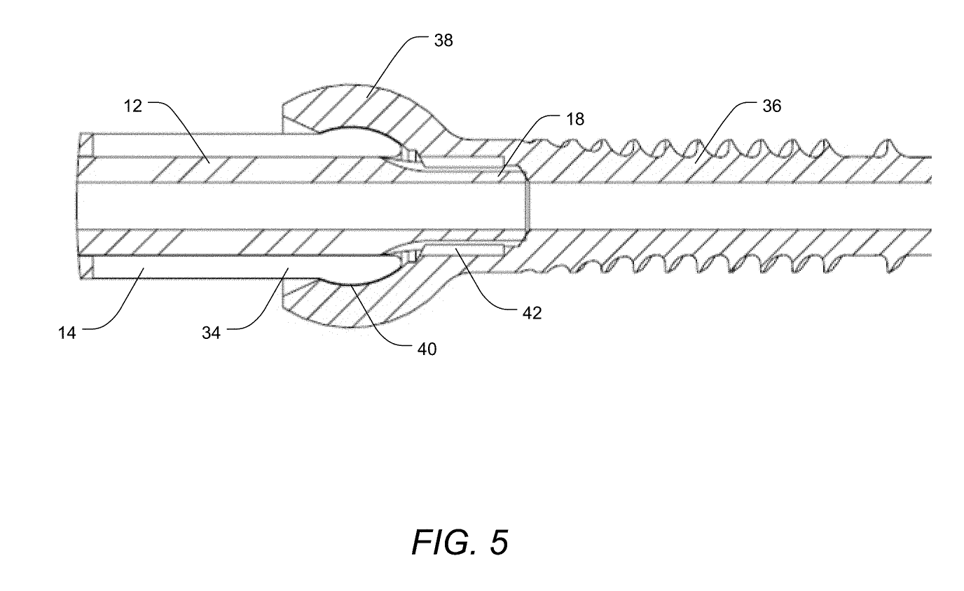

As another example, the inner sleeve 12 may be advanced still further within the outer sleeve 14, until the tip of the distal end 18 of the inner sleeve 12 enters into and mates with the driving feature 42 of the surgical screw 36, as illustrated in FIG. 5. The fit between the tip of the distal end 18 of the inner sleeve 12 and the driving feature 36 need not be overly snug, as the surgical screw 36 remains retained by the outward displacement of the fingers 34 which remains in place by the presence of the inner sleeve 12 within the fingers. Instead, the transfer of rotational torque occurs solely at the engagement of the tip of the distal end 18 of the inner sleeve and the driving feature 42.

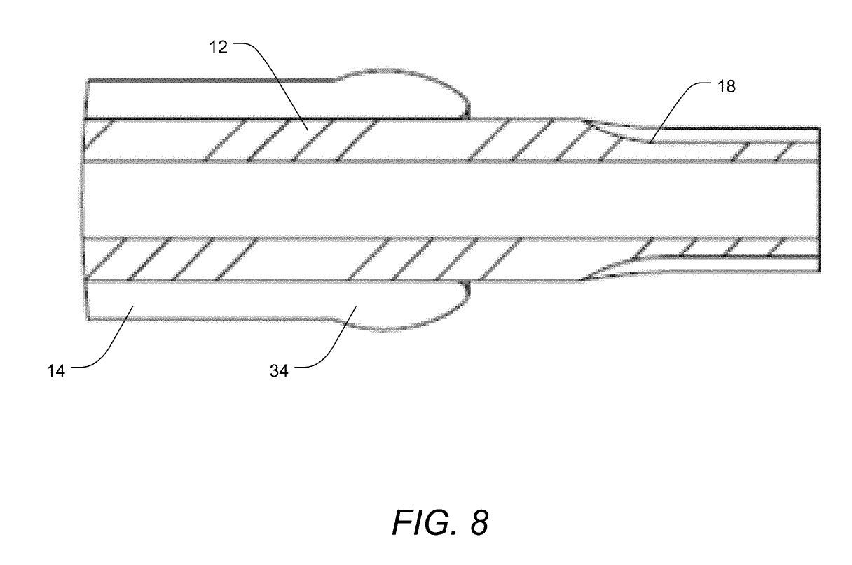

FIG. 8 illustrates that the screwdriver 10 may be used as a standard screwdriver without engaging the self-retaining features. In such use, the inner sleeve 12 is advanced fully within the outer sleeve 14, until the distal end 18 advances past the fingers 34 of the outer sleeve, thereby exposing the tip of the distal end 18, which can be used to mate with and drive a compatible surgical screw or other fastener. In some embodiments, the inner sleeve 12 may be capable of advancing within the outer sleeve 14 to an extent greater than is necessary for use with a retained surgical screw, to better expose the tip of the distal end 18 of the inner sleeve 12 for use as a standard screwdriver. If desired, multiple detents 24 may be provided to retain the inner sleeve 12 at a desired stage of advancement within the outer sleeve 14 to permit use of the screwdriver 10 as a self-retaining screwdriver or alternatively as a standard, non-retaining, screwdriver.

When the screwdriver 10 is to be used as a retaining screwdriver, the inner sleeve 12 is withdrawn from the outer sleeve 14 until the fingers 34 are free to flex sufficiently to permit entry of the fingers into the head 38 of the surgical screw 36. The surgical screw 36 is placed on the distal end 22 of the outer sleeve 14, which forces the fingers inward as the head 38 pushes over the enlarged portion of the fingers 34. Then the inner sleeve 12 is advanced within the outer sleeve 14 until the tip of the distal end 18 engages the driving feature 42 of the surgical screw 36. If necessary, the surgical screw 36 may be rotated on the fingers 34 as this occurs to ensure a proper rotational orientation of the surgical screw 36 relative to the inner sleeve 12 for mating of the tip of the distal end 18 with the driving feature 42. At this point, the surgical screw 36 is retained on the screw driver 10, and is prepared for delivery to the surgical site, either by visual placement or using a guide wire that extends through the surgical screw 36 and the hollow inner sleeve 12.

The interaction between the protrusion 26 and the detent 24 ensures that the inner sleeve 12 does not withdraw from the outer sleeve 14 except when desired by the surgeon. The screwdriver 10 is then used to advance the surgical screw 36 into the surgical site (e.g. bone such as a pedicle of a vertebrae) a desired amount. As the surgical screw 36 is retained on the screwdriver 10 using a purely internal securement, the surgeon's view is relatively unobstructed, which may assist in proper placement of the surgical screw 36. Once the surgical screw 36 is fully placed, the inner sleeve 12 is withdrawn from the outer sleeve 14 an amount sufficient to permit the fingers 34 to again flex inward as the screwdriver 10 is withdrawn from the surgical screw 36.

If necessary, the surgical screw 36 can be readily re-engaged by the screwdriver 10 in situ, simply by repeating the processes discussed above. Accordingly, the surgeon can readily disengage the screwdriver 10 to check placement, and can readily re-engage the screwdriver 10 to further advance the surgical screw 36, to remove the surgical screw 36, or to otherwise adjust placement of the surgical screw 36, without worrying about losing the surgical screw 36 at the surgical site.

All components of the screwdriver 10 may be manufactured of readily sterilizable materials such as steel, titanium, and various alloys of these materials or other metal materials. Accordingly, the screwdriver 10 may be easily sterilized without concerns of reducing the effectiveness of the engagement between the screwdriver 10 and the surgical screw 36. Because the deformation of the fingers 34 is elastic, and because the inner sleeve 12 serves to physically prevent inward motion of the fingers 34 once the inner sleeve 12 is advanced within the outer sleeve 14, the retaining features of the screwdriver 10 are highly durable and any wear effects are minimal at most.

While embodiments of the screwdriver 10 are anticipated to be of particular use in the surgical field, the embodiments of the invention are not limited to such field. Accordingly, embodiments of the screwdriver 10 may be used in any situation where a screw that can have a head adapted to include a partially spherical receptacle can be used.

The present invention may be embodied in other specific forms without departing from its spirit or essential characteristics. The described embodiments are to be considered in all respects only as illustrative and not restrictive. The scope of the invention is, therefore, indicated by the appended claims, rather than by the foregoing description. All changes which come within the meaning and range of equivalency of the claims are to be embraced within their scope.

What is claimed and desired to be secured by Letters Patent is:

1. A self-retaining screwdriver comprising:
an elongate inner driver rod; and
an outer sleeve adapted to be disposed about the inner driver rod and comprising a distal end, the distal end of the outer sleeve comprising a plurality of fingers separated by longitudinal slots in the outer sleeve.

2. The self-retaining screwdriver as recited in claim 1, wherein each of the fingers comprises a retention feature.

3. The self-retaining screwdriver as recited in claim 2, wherein the retention feature comprises an area of thickening of the finger, whereby an effective diameter of the outer sleeve at the retention features of the fingers is increased.

4. The self-retaining screwdriver as recited in claim 3, wherein when the inner driver rod is disposed within the outer sleeve with a distal portion of the inner driver rod extended as far as the retention feature, the presence of the inner driver rod prevents the fingers from being radially displaced inwardly.

5. The self-retaining screwdriver as recited in claim 4, wherein when the inner driver rod is at least partially withdrawn proximally within the outer sleeve such that the distal portion of the inner driver rod does not extend to a distal end of the fingers, the fingers can be radially displaced inwardly, effectively decreasing the diameter of the outer sleeve at the retention features of the fingers.

6. The self-retaining screwdriver as recited in claim 1, wherein the inner driver rod comprises a distal end comprising a driver feature adapted to engage a screw and to transfer rotational torque from the inner driver rod to the screw.

7. The self-retaining screwdriver as recited in claim 1, wherein the outer sleeve comprises a longitudinal alignment slot, and wherein the inner driver rod comprises a protrusion adapted to be received within the longitudinal alignment slot of the outer sleeve.

8. The self-retaining screwdriver as recited in claim 7, wherein the protrusion and the alignment slot permit transmission of rotational torque from the inner driver rod to the outer sleeve or from the outer sleeve to the inner driver rod.

9. The self-retaining screwdriver as recited in claim 1, further comprising a detent that limits unintentional retraction of the inner driver rod from the outer sleeve.

10. The self-retaining screwdriver as recited in claim 1, wherein the elongate inner driver rod is hollow to permit passage of a guide wire therethrough.

11. A self-retaining screwdriver comprising:
an elongate inner driver rod; and
an outer sleeve adapted to be disposed about the inner driver rod and comprising a distal end, the distal end of the outer sleeve comprising a plurality of fingers separated by longitudinal slots in the outer sleeve, and wherein each of the fingers comprises a retention feature.

12. The self-retaining screwdriver as recited in claim 11, wherein the retention feature comprises an area of thickening of the finger, whereby an effective diameter of the outer sleeve at the retention features of the fingers is increased.

13. The self-retaining screwdriver as recited in claim 12, wherein when the inner driver rod is disposed within the outer sleeve with a distal portion of the inner driver rod extended as far as the retention feature, the presence of the inner driver rod prevents the fingers from being radially displaced inwardly.

14. The self-retaining screwdriver as recited in claim 13, wherein when the inner driver rod is at least partially withdrawn proximally within the outer sleeve such that the distal portion of the inner driver rod does not extend to a distal end of the fingers, the fingers can be radially displaced inwardly, effectively decreasing the diameter of the outer sleeve at the retention features of the fingers.

15. The self-retaining screwdriver as recited in claim 11, wherein the inner driver rod comprises a distal end comprising a driver feature adapted to engage a screw and to transfer rotational torque from the inner driver rod to the screw.

16. The self-retaining screwdriver as recited in claim 11, wherein the outer sleeve comprises a longitudinal alignment slot, and wherein the inner driver rod comprises a protrusion adapted to be received within the longitudinal alignment slot of the outer sleeve.

17. The self-retaining screwdriver as recited in claim 16, wherein the protrusion and the alignment slot permit transmission of rotational torque from the inner driver rod to the outer sleeve or from the outer sleeve to the inner driver rod.

18. The self-retaining screwdriver as recited in claim 11, further comprising a detent that limits unintentional retraction of the inner driver rod from the outer sleeve.

19. A method for preparing a self-retaining screw driver with a surgical screw retained thereon ready for use in a surgical procedure, the method comprising:
providing an outer sleeve having a distal end comprising a plurality of fingers separated by longitudinal slots in the outer sleeve;
advancing the outer sleeve into a partially spherical receptacle of a surgical screw, whereby the fingers are temporarily and elastically forced radially inward as the surgical screw is received on the outer sleeve;
advancing an inner sleeve into the outer sleeve until a distal end of the inner sleeve is disposed within the fingers, thereby forcing the fingers radially outwardly and preventing the fingers from being displaced radially inwardly, thereby retaining the surgical screw on the outer sleeve.

20. The method of claim 19, further comprising advancing the inner sleeve further within the outer sleeve until a tip of the distal end of the inner sleeve protrudes beyond the outer sleeve and mates with and engages a driving feature of the surgical screw located more distal than the partially spherical receptacle.
